(19) United States
(12) Patent Application Publication
(10) Pub. No.: US 2006/0194991 A1
(43) Pub. Date: Aug. 31, 2006

(54) Treatment of alkylation catalyst poisons

(75) Inventors: James R. Butler, League City, TX (US); Jim Merrill, Katy, TX (US); Marcus Ledoux, Baton Rouge, LA (US); Michael Betbeze, Baton Rouge, LA (US)

Correspondence Address: David J. Alexander, P.O. Box 674412, Houston, TX, 77167, US

(73) Assignee: Fina Technology, Inc., Houston, TX (US)

(21) Appl. No.: 11/326,666

(22) Filed: Jan. 7, 2006

Related U.S. Application Data
(60) Provisional application No. 60/656,331, filed on Feb. 25, 2005.

(51) Int. Cl. C07C 2/00

(52) U.S. Cl. 585/323

(57) ABSTRACT

Alkylation processes are described herein. The alkylation process generally includes contacting an input stream including benzene with an alkylation catalyst and an alkylating agent to form an alkylation output stream including ethylbenzene. The alkylation process further includes contacting at least a portion of the alkylation output stream with a transalkylation catalyst and a benzene source to form a transalkylation output stream, wherein the benzene source is selected to minimize the amount of alkylation catalyst poisons contacting the alkylation catalyst.

### CROSS-REFERENCE TO RELATED APPLICATIONS

This application claims the benefit of U.S. Provisional Patent Application Ser. No. 60/656,331, filed Feb. 25, 2005.

### FIELD

Embodiments of the present invention generally relate to alkylation systems. In particular, embodiments of the present invention relate to minimizing alkylation catalyst deactivation.

### BACKGROUND

Alkylation processes used to form ethylbenzene generally include contacting an input stream with an alkylation catalyst and an alkylating agent to for the ethylbenzene. The input stream generally includes benzene. At least a portion of the benzene may be supplied from the output of dehydrogenation systems used to form styrene.

However, such benzene may include alkylation catalyst poisons (e.g., nitrogen containing compounds used as additives in the dehydrogenation process), which results in frequent alkylation catalyst replacement or regeneration.

Therefore, a need exists to cost effectively supply benzene to alkylation systems while minimizing the amount of alkylation catalyst poisons included therein.

### SUMMARY

Embodiments of the present invention include an alkylation process. The alkylation process generally includes contacting an input stream including benzene with an alkylation catalyst and an alkylating agent to form an alkylation output stream including ethylbenzene. The alkylation process further includes contacting at least a portion of the alkylation output stream with a transalkylation catalyst and a benzene source to form a transalkylation output stream, wherein the benzene source is selected to minimize the amount of alkylation catalyst poisons contacting the alkylation catalyst.

Embodiments of the invention further include a method of reducing alkylation catalyst deactivation. The method generally includes supplying benzene to an alkylation system including an alkylation catalyst disposed therein, wherein at least a portion of the benzene is supplied from a transalkylation system output stream.

### BRIEF DESCRIPTION OF DRAWINGS

FIG. 1 illustrates a dehydrogenation system.

FIG. 2 (Prior Art) illustrates an alkylation system.

FIG. 3 illustrates an embodiment of an alkylation system.

### DETAILED DESCRIPTION

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions, when the information in this patent is combined with available information and technology. Various terms as used herein are shown below. To the extent a term used in a claim is not defined below, it should be given the broadest definition persons in the pertinent art have given that term as reflected in printed publications and issued patents.

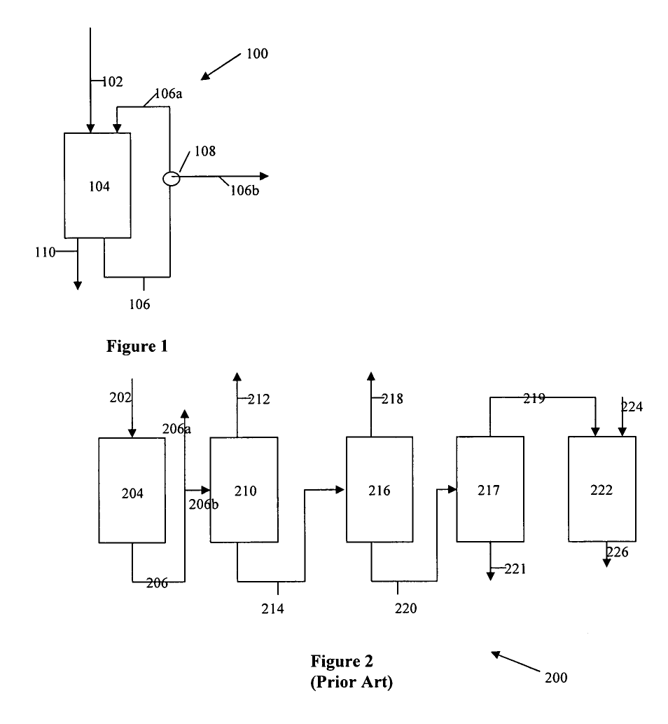

FIG. 1 (Prior Art) illustrates an embodiment of a catalytic dehydrogenation/purification process 100. Dehydrogenation processes generally include contacting an alkyl aromatic hydrocarbon with a dehydrogenation catalyst to form a vinyl aromatic hydrocarbon. A variety of catalysts can be used in the catalytic dehydrogenation process and are known to one skilled in the art, such as potassium iron oxide catalysts and cesium iron oxide catalysts.

In FIG. 1, an input stream 102 is supplied to a dehydrogenation system 104. As used herein, individual streams will be denoted with a number, but it is generally known that such streams flow through conduits, such as pipes. The input stream 102 includes an alkyl aromatic hydrocarbon, such as ethylbenzene, for example. Steam may further be added to the input stream 102. The steam may be added to the input stream 102 in any manner known to one skilled in the art. Although the amount of steam contacting the input stream 102 is determined by individual process parameters, the input stream 102 may have a steam to alkyl aromatic hydrocarbon weight ratio of from about 0.01:1 to about 15:1, or from about 0.3:1 to about 10:1, or from about 0.6:1 to about 3:1 or from about 1:1 to about 2:1, for example.

The dehydrogenation system 104 may include any reaction vessel, combination of reaction vessels and/or number of reaction vessels (either in parallel or in series) known to one skilled in the art for the conversion of an alkyl aromatic hydrocarbon to a vinyl aromatic hydrocarbon. For example, the one or more reaction vessels may be fixed bed vessels, fluidized bed vessels and/or tubular reactor vessels.

The dehydrogenation processes discussed herein are generally high temperature processes. As used herein, the term “high temperature” refers to process operation temperatures, such as reaction vessel and/or process line temperatures (e.g., the temperature of the input stream 102 at the vessel inlet, not shown) of from about 150° C. to about 1000° C., or from about 300° C. to about 800° C., or from about 500° C. to about 700° C. or from about 550° C. to about 650° C., for example.

The output 106 from the dehydrogenation system 104 (e.g., ethylbenzene and styrene) may be supplied to a splitter 108 where the output 106 is separated into at least two portions. A first portion 106a of the output 106 may be recycled back to the dehydrogenation system 104 (not shown). The first portion 106a may include unreacted ethylbenzene, for example. A second portion 106b of the dehydrogenation product may be supplied to an alkylation/transalkylation process, described in more detail below. The second portion 106b generally includes benzene and may further include toluene, for example. Styrene product 110 may be recovered and used for any suitable purpose, such as the production of polystyrene, for example. Although shown as a separate line in FIG. 1, the styrene may be recovered from the dehydrogenation system 104 via the same line conduit 106 as the first and second portions (not shown), for example.

FIG. 2 (Prior Art) illustrates an embodiment of an alkylation/transalkylation process 200. The alkylation and transalkylation processes generally include contacting an input, such as benzene and/or diethylbenzene with a catalyst for the recovery of ethylbenzene. A variety of catalysts can be used in the alkylation/transalkylation process 200 and are known to ones skilled in the art, such as acidic zeolite catalysts (e.g., zeolite beta catalysts and zeolite Y catalysts.)

In FIG. 2, an input stream 202 is supplied to an alkylation system 204. The input stream 202 may include benzene and ethylene from a variety of sources. For example, the input stream 202 may be fed from a fresh benzene source, a fresh ethylene source and/or a variety of recycle sources. As used herein, the term “fresh benzene” refers to a source having about 95 wt. % or more benzene, or about 98 wt. % or more benzene or about 99 wt. % or more benzene, for example. The benzene sources may further include ethylbenzene, non-aromatics and/or toluene, for example. As used herein, the term “recycle” refers to an output of a system, such as an alkylation system, that is then returned as input to either that same system or another system within the process. In one embodiment, the molar ratio of benzene to ethylene in the input stream 202 may be from about 1:1 to about 30:1, or from about 1:1 to about 20:1 or from about 1:1 to about 15:1, for example.

The alkylation system 204 may include any reaction vessel, combination of reaction vessels and/or number of reaction vessels (either in parallel or in series) known to one skilled in the art. Such reaction vessels may be vapor phase or liquid phase reactors that may be operated at reactor temperatures and pressures sufficient to maintain the alkylation reaction in the supercritical phase, e.g., the benzene is in the supercritical state, or in the liquid phase, as determined by individual process parameters.

A first portion 206a of the output 206 from the alkylation system 204 may be recycled back to the alkylation system 204 or recovered for other purposes. The first portion may include benzene, for example. A second portion 206b of the output 206 may be supplied to a benzene separation system 210. The second portion 206b may include ethylbenzene, for example.

The benzene separation system 210 may include any process known to one skilled in the art, for example, one or more distillation columns, either in series or in parallel. Benzene product 212 may be recovered and recycled back to the alkylation system 204 or used for any other purpose. The benzene may be recycled back to the alkylation system 204 in any way known to one skilled in the art, for example, by combining the benzene 212 with the input stream 202 or by directly feeding the benzene 212 into the alkylation system 204. The bottoms fraction 214 from the benzene separation system 210 may be supplied to an ethylbenzene separation system 216. The bottoms fraction 214 may include ethylbenzene and/or polyalkylated benzenes, such as polyethylbenzene (PEB), for example.

The ethylbenzene separation system 216 may include any process known to one skilled in the art, for example, one or more distillation columns, either in series or in parallel. Ethylbenzene product 218 may be recovered and used for any suitable purpose, such as the production of vinyl benzene or styrene, for example. In one embodiment, the ethylbenzene 218 is fed to the dehydrogenation process 100, e.g., input 102. The bottoms fraction 220 of the ethylbenzene separation system 216 may be supplied to a polyethylbenzene (PEB) separation system 217. The bottoms fraction 220 may include polyethylbenzenes, such as diethylbenzene and heavier aromatics (e.g., cumene and butylbenzene,) for example.

The PEB separation system 217 may include any process known to one skilled in the art, for example, one or more distillation columns, either in series or in parallel. Product 219 may be recovered from the PEB separation system 217 and may be supplied to a transalkylation system 222. The product 219 may include diethylbenzene and liquid phase triethylbenzene, for example. Heavies 221 may further be recovered from the PEB separation system 217 for further processing and recovery (not shown).

The transalkylation system 222 may include any reaction vessel, combination of reaction vessels and/or number of reaction vessels (either in parallel or in series) known to one skilled in the art.) In one embodiment, the transalkylation system 222 is operated under liquid phase conditions. In one embodiment the transalkylation catalyst has a somewhat larger pore size than the molecular sieve catalyst used in the alkylation system reactor(s).

In addition to product 219, benzene 224 may be supplied to the transalkylation system 222. The output 226 from the transalkylation system 222 may be recycled to the benzene separation system 210 (not shown) or used for any other purpose. The output 226 may be fed to the benzene separation system 210 in any way known to one skilled in the art, for example, by combining the output 226 with line 206b or by directly feeding the output into the benzene separation system 210.

Referring back to FIG. 1, dehydrogenation processes 100 may include the addition of nitrogen containing compounds (not shown.) The nitrogen containing compounds, such as amines, may be added to the dehydrogenation process 100 for a variety of purposes, such as polymerization inhibitors and/or neutralizers, for example. Therefore, the second portion of the dehydrogenation product 106b may include such nitrogen compounds. For example, in one embodiment, the second portion 106b may include as much as 1 ppm of nitrogen containing compounds.

In many processes, such as that shown in FIG. 2, the second portion of the dehydrogenation product 106b is fed to the alkylation/transalkylation process 200 for further processing. Generally, the second portion 106b is fed to the alkylation system 204, either via its own inlet (not shown) or in combination with fresh benzene 202 and/or recycled benzene 206a from the alkylation system. However, the nitrogen compounds present in the second portion 106b may poison the alkylation catalyst, therefore requiring more frequent regeneration and/or replacement of such catalyst. In one embodiment, the amount of poisons entering the alkylation system is less than about 50 ppb, or less than about 40 ppb, or less than 30 ppb or less than 20 ppb, for example.

Embodiments of the present invention seek to reduce the poison effect of the nitrogen containing compounds in the second portion of the dehydrogenation product 106b.

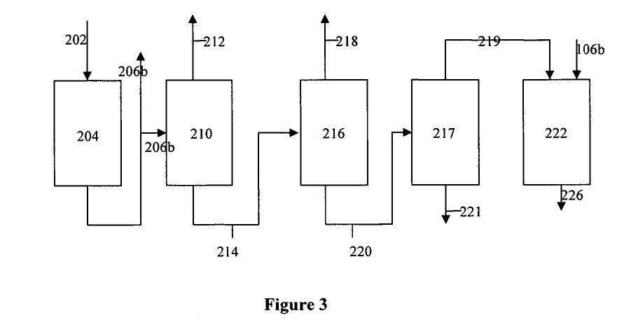

In one embodiment, the poison effect is reduced via the process illustrated in FIG. 3. FIG. 3 illustrates an embodiment wherein the second portion of the dehydrogenation product 106b is fed to the transalkylation system 222 for further processing. The second portion 106b may be fed to the transalkylation system 222 via a variety of methods, such as combining the second portion with lines 219 and/or 224 (not shown) or by directly feeding the second portion into the transalkylation system 222. Such an embodiment has been demonstrated to reduce the amount of nitrogen containing compounds entering the alkylation system by about 50%. Preferably, the amount is reduced by at least 20%, or about 30% or about 40%, for example.

In another embodiment, the poison effect is reduced by passing at least a portion of line 206a (not shown) to the transalkylation system. Generally, nitrogen compounds, along with other poisons, present in the input stream pass through the first separation system resulting in an overhead product including such compounds. Therefore, it is contemplated to pass at least a portion of such overhead product to the transalkylation system. The at least a portion of line 206a may be at least 10 percent, or at least 20 percent or at least 30 percent thereof, for example. Such process stream flow reduced the amount of poisons contacting the alkylation catalyst.

Although not shown in the Figures, additional process equipment, such as heat exchangers, may be employed throughout the process shown above and such placement is generally known to one skilled in the art.

While the foregoing is directed to embodiments of the present invention, other and further embodiments of the invention may be devised without departing from the basic scope thereof and the scope thereof is determined by the claims that follow.

What is claimed is:

1. An alkylation process comprising:
contacting an input stream comprising benzene with an alkylation catalyst and an alkylating agent to form an alkylation output stream comprising ethylbenzene; and
contacting at least a portion of the alkylation output stream with a transalkylation catalyst and a benzene source to form a transalkylation output stream, wherein the benzene source is selected to minimize the amount of alkylation catalyst poisons contacting the alkylation catalyst.

2. The alkylation process of claim 1, wherein the benzene source is benzene obtained from a dehydrogenation system output.

3. The alkylation process of claim 2, wherein the input stream is free from dehydrogenation system output.

4. The alkylation process of claim 1, wherein the input stream comprises at least a portion of the alkylation output stream.

5. The alkylation process of claim 1, wherein the catalyst poisons are selected from nitrogen, sulfur and oxygen containing compounds and combinations thereof.

6. The alkylation process of claim 1, wherein the benzene source comprises recycle benzene.

7. The alkylation process of claim 6, wherein the recycle benzene comprises at least a portion of the alkylation output stream.

8. The alkylation process of claim 1, wherein the input stream comprises fresh benzene.

9. The alkylation process of claim 5, wherein the about 50 ppb or less of the catalyst poisons contact the alkylation catalyst.

10. The alkylation process of claim 1, wherein the catalyst poisons comprise about 1 ppm or less nitrogen.

11. A method of reducing alkylation catalyst deactivation comprising:
providing an alkylation system having an alkylation catalyst disposed therein;
contacting the alkylation catalyst with ethylene; and
supplying benzene to the alkylation system to contact the alkylation catalyst, wherein at least a portion of the benzene is supplied from a transalkylation system output stream.

12. The method of claim 11, wherein the benzene is supplied from at least one source, wherein the at least one source is the transalkylation system output stream and any additional sources are selected from a separation system output stream, fresh benzene and combinations thereof.

13. The method of claim 12, wherein the additional source consists essentially of fresh benzene.

14. The method of claim 11, wherein the benzene is essentially free of recycled benzene.

15. The method of claim 14, wherein the benzene comprises about 50 ppb or less poisons.

16. The method of claim 11, wherein the molar ratio of benzene to ethylene in the alkylation system is from about 1:1 to about 30:1.
